(19) United States
(12) Patent Application Publication
(10) Pub. No.: US 2019/0192719 A1
(43) Pub. Date: Jun. 27, 2019

(54) Fan Mountable Air Freshening Device

(71) Applicants: Ronald Drayton, Hephzibah, GA (US); Ureatha Drayton, Hephzibah, GA (US)

(72) Inventors: Ronald Drayton, Hephzibah, GA (US); Ureatha Drayton, Hephzibah, GA (US)

(21) Appl. No.: 15/850,735

(22) Filed: Dec. 21, 2017

(51) Int. Cl. A61L 9/12

(52) U.S. Cl. CPC A61L 9/122; A61L 2209/15

(57) ABSTRACT

A fan mountable air freshening device for releasing fragrance into circulated air includes a plurality of pads that is configured to couple to a fan. The pads are impregnated with a volatilizable substance. The pads are configured to release the volatilizable substance into air that is circulated by the fan.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to air freshening devices and more particularly pertains to a new air freshening device for releasing fragrance into circulated air.

### BRIEF SUMMARY OF THE INVENTION

An embodiment of the disclosure meets the needs presented above by generally comprising a plurality of pads that is configured to couple to a fan. The pads are impregnated with a volatilizable substance. The pads are configured to release the volatilizable substance into air that is circulated by the fan.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a fan mountable air freshening device according to an embodiment of the disclosure.

FIG. 2 is a bottom view of an embodiment of the disclosure.

FIG. 3 is a cross-sectional view of an embodiment of the disclosure.

FIG. 4 is a side view of an embodiment of the disclosure.

FIG. 5 is an in-use view of an embodiment of the disclosure.

### DETAILED DESCRIPTION OF THE INVENTION

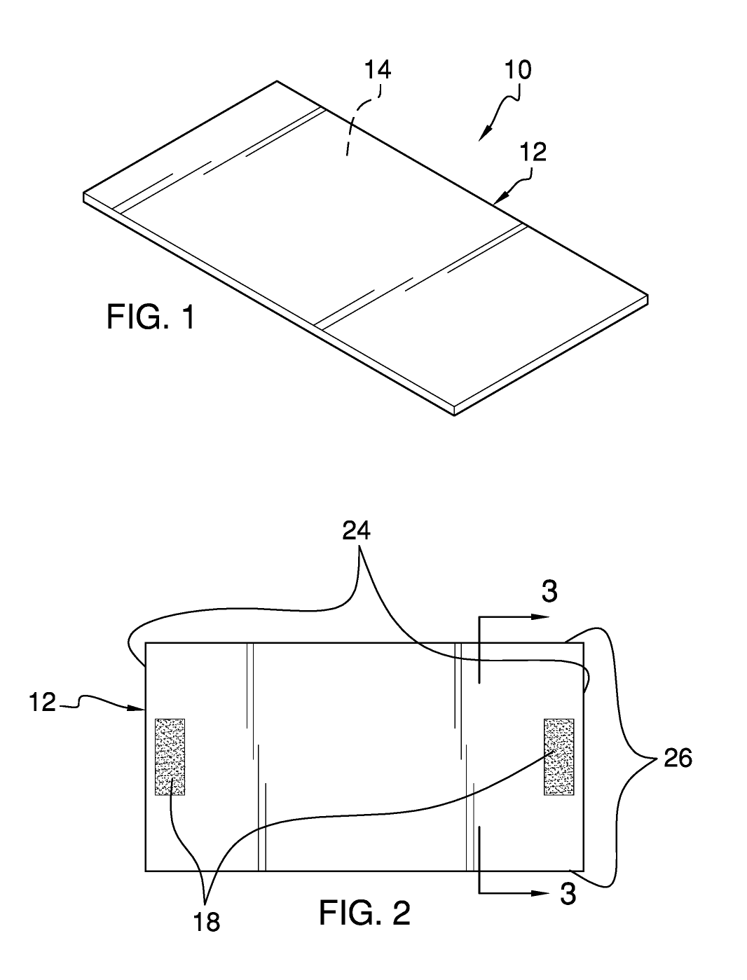

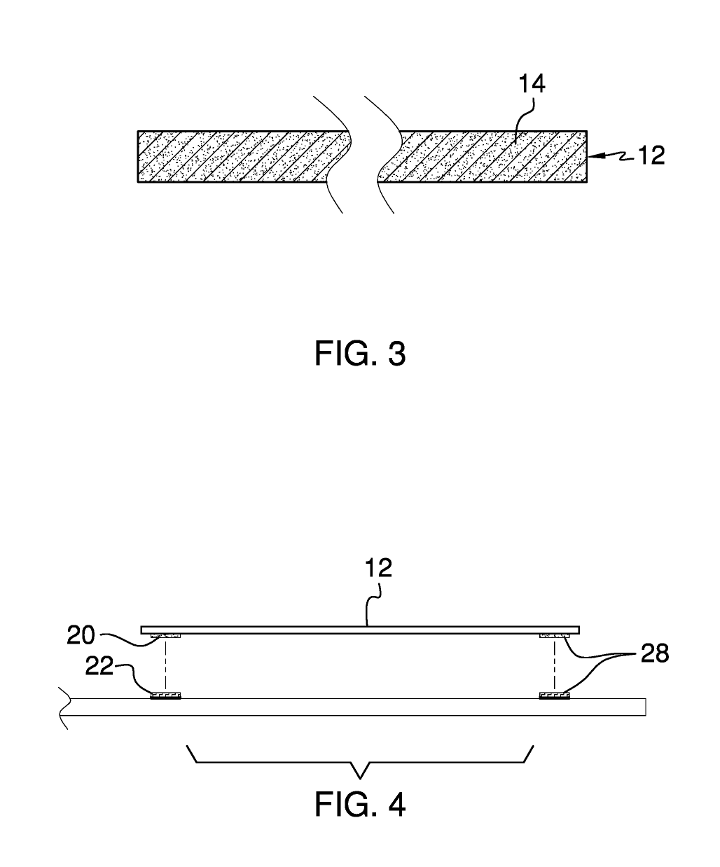

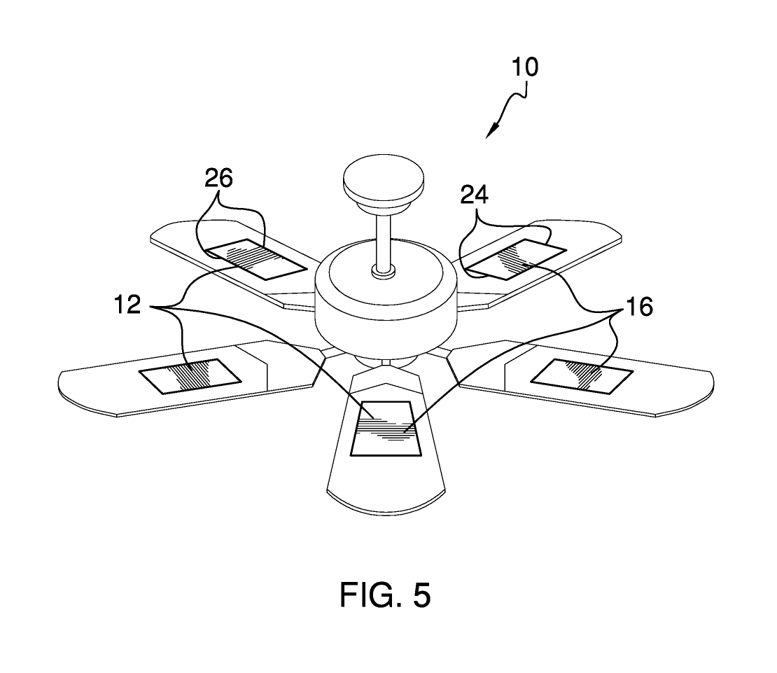

With reference now to the drawings, and in particular to FIGS. 1 through 5 thereof, a new air freshening device embodying the principles and concepts of an embodiment of the disclosure and generally designated by the reference numeral 10 will be described.

As best illustrated in FIGS. 1 through 5, the fan mountable air freshening device 10 generally a plurality of pads 12 that is configured to couple to a fan. The pads 12 are impregnated with a volatilizable substance 14. The pads 12 are configured to release the volatilizable substance 14 into air that is circulated by the fan.

In one embodiment, the pads 12 are configured to be impregnated with the volatilizable substance 14. A user is positioned to impregnate the pads 12 with a respective volatilizable substance 14 that selected from a plurality of compounds 16. The plurality of compounds 16 can include fragrances, such as lavender, sea breeze, vanilla, cherry, pine, apple, cinnamon, rose, and lilac. The plurality of compounds 16 also can include compounds 16 having medicinal properties, such as menthol and eucalyptus oil, to improve the breathing of persons proximate to the fan.

The pads 12 are configured to couple to a blade of a ceiling fan. The pads 12 are configured to couple to a control chain of the ceiling fan. The pads 12 are substantially rectangularly shaped and comprise cloth which may be a non-woven polyester. Alternatively, the pads 12 are comprised of paper which may be card stock.

Each pad 12 comprises a plurality of connectors 18. The connectors 18 are configured to couple to the fan to couple the pad 12 to the fan. In one embodiment, each connector 18 comprises a first fastener 20 and a second fastener 22. The first fastener 20 is coupled to the pad 12. The second fastener 22 is configured to couple to the fan. The second fastener 22 is complementary to the first fastener 20. Each second fastener 22 is positioned to couple to a respective first fastener 20 to couple the pad 12 to the fan.

The second fastener 22 is configured to adhesively couple to the fan. The plurality of connectors 18 comprises two connectors 18 that are positioned singly proximate to opposing ends 24 of the pad 12. The connectors 18 are positioned equally distant from opposing sides 26 of the pad 12. Each second fastener 22 and the respective first fastener 20 comprise a hook and loop fastener 28.

In use, the user is positioned to impregnate the pads 12 with the respective volatilizable substance 14 that is selected from the plurality of compounds 16. Each second fastener 22 is positioned to couple to the respective first fastener 20 to couple the pad 12 to the fan. The pads 12 are configured to release the respective volatilizable substance 14 into the air that is circulated by the fan.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A fan mountable air freshening device comprising:
a plurality of pads configured for coupling to a fan, said pads being impregnated with a volatilizable substance; and
wherein said pads are configured for coupling to the fan such that said pads are configured for releasing said volatilizable substance into air being circulated by the fan.

2. The device of claim 1, further including said pads being configured for impregnating with said volatilizable substance such that a user is positioned for impregnating said pads with a respective said volatilizable substance selected from a plurality of compounds.

3. The device of claim 1, further including said pads being configured for coupling to a blade of a ceiling fan.

4. The device of claim 1, further including said pads being configured for coupling to a control chain of a ceiling fan.

5. The device of claim 1, further including said pads being substantially rectangularly shaped.

6. The device of claim 1, further including said pads comprising cloth.

7. The device of claim 6, further including said pads comprising non-woven polyester.

8. The device of claim 1, further including said pads comprising paper.

9. The device of claim 8, further including said pads comprising card stock.

10. The device of claim 1, further including each said pad comprising a plurality of connectors, said connectors being configured for coupling to the fan for coupling said pad to the fan, each said connector comprising a first fastener and a second fastener, said first fastener being coupled to said pad, said second fastener being configured for coupling to the fan, said second fastener being complementary to said first fastener, wherein said second fasteners are positioned on the fan such that each said second fastener is positioned for coupling to a respective said first fastener for coupling said pad to the fan.

11. The device of claim 10, further including said second fastener being configured for adhesively coupling to the fan.

12. The device of claim 10, further including said plurality of connectors comprising two said connectors positioned singly proximate to opposing ends of said pad.

13. The device of claim 12, further including said connectors being positioned equally distant from opposing sides of said pad.

14. The device of claim 10, further including each said second fastener and said respective said first fastener comprising a hook and loop fastener.

15. A fan mountable air freshening device comprising:
a plurality of pads configured for coupling to a fan, said pads being impregnated with a volatilizable substance, wherein said pads are configured for coupling to the fan such that said pads are configured for releasing said volatilizable substance into air being circulated by the fan, said pads being configured for impregnating with said volatilizable substance such that a user is positioned for impregnating said pads with a respective said volatilizable substance selected from a plurality of compounds, said pads being configured for coupling to a blade of a ceiling fan, said pads being configured for coupling to a control chain of the ceiling fan, said pads being substantially rectangularly shaped, said pads comprising cloth, said pads comprising non-woven polyester, said pads comprising paper, said pads comprising card stock;
each said pad comprising a plurality of connectors, said connectors being configured for coupling to the fan for coupling said pad to the fan, each said connector comprising a first fastener and a second fastener, said first fastener being coupled to said pad, said second fastener being configured for coupling to the fan, said second fastener being complementary to said first fastener, wherein said second fasteners are positioned on the fan such that each said second fastener is positioned for coupling to a respective said first fastener for coupling said pad to the fan, said second fastener being configured for adhesively coupling to the fan, said plurality of connectors comprising two said connectors positioned singly proximate to opposing ends of said pad, said connectors being positioned equally distant from opposing sides of said pad, each said second fastener and said respective said first fastener comprising a hook and loop fastener; and
wherein said pads are configured for impregnating with said volatilizable substance such that the user is positioned for impregnating said pads with said respective said volatilizable substance selected from said plurality of compounds, wherein said second fasteners are positioned on the fan such that each said second fastener is positioned for coupling to said respective said first fastener for coupling said pad to the fan such that said pads are configured for releasing said respective said volatilizable substance into the air being circulated by the fan.
